(19) United States
(12) Patent Application Publication
(10) Pub. No.: US 2026/0358251 A1
(43) Pub. Date: Nov. 12, 2026

(54) ANTIBODY CONCENTRATION MEASUREMENT

(71) Applicant: Beckman Coulter, Inc., Brea, CA (US)

(72) Inventors: Julie Lutti, St. Petersburg, FL (US); Todd Halvorson, Ft. Collins, CO (US)

(73) Assignee: Beckman Coulter, Inc., Brea, CA (US)

(21) Appl. No.: 19/475,282

(22) PCT Filed: Apr. 12, 2024
(86) PCT No.: PCT/US2024/024315

§ 371 Date: Oct. 14, 2025

Related U.S. Application Data
(60) Provisional application No. 63/496,221, filed on Apr. 14, 2023.

(51) Int. Cl. G01N 21/64; G01N 33/68

(52) U.S. Cl. CPC G01N 21/6428; G01N 33/6854; G01N 2021/6439

(57) ABSTRACT

A method of fluorescence polarization is disclosed for measuring a concentration of an antibody in a sample. The method includes performing a measurement cycle measuring a first set of sampling phases of fluorescence polarized in a first polarization direction and a second set of sampling phases of the fluorescence polarized in a second polarization direction. At least one sampling phase of the fluorescence polarized in the second polarization direction occurs between sampling phases of the fluorescence polarized in the first polarization direction. The method includes calculating a first value of the first set of sampling phases and calculating a second value of the second set of sampling phases. The method includes determining the concentration of the antibody based on a function of the first and second values.

### CROSS-REFERENCE TO RELATED APPLICATION

This application is being filed on Apr. 12, 2024, as a PCT International application and claims the benefit of and priority to U.S. Provisional Application No. 63/496,221, filed Apr. 14, 2023, the disclosure of which is hereby incorporated by reference in its entirety.

### BACKGROUND

An antibody is a large, Y-shaped protein used by the immune system to identify and neutralize foreign objects such as pathogenic bacteria and viruses. The antibody recognizes a unique molecule of the pathogen, called an antigen. Each tip of the Y-shaped antibody contains a paratope that matches an epitope on the antigen, allowing the antibody to bind with the antigen. Using this binding mechanism, an antibody can tag a microbe or an infected cell for attack by other parts of the immune system, or can neutralize the microbe or the infected cell directly. Immunoglobulin G (IgG) is the most common type of antibody found in blood circulation. A concentration of an antibody in a cell sample can be measured by fluorescence polarization.

### SUMMARY

In general terms, the present disclosure relates to measuring a concentration of an antibody in a cell sample by fluorescence polarization. In one possible configuration, a measurement cycle is performed to reduce errors that can result from signal intensity variations and noise during the measurement. Various aspects are described in this disclosure, which include, but are not limited to, the following aspects.

One aspect relates to a method of fluorescence polarization for measuring a concentration of an antibody in a sample, the method comprising: performing a measurement cycle measuring a first set of sampling phases of fluorescence polarized in a first polarization direction and a second set of sampling phases of the fluorescence polarized in a second polarization direction, at least one sampling phase of the fluorescence polarized in the second polarization direction occurring between sampling phases of the fluorescence polarized in the first polarization direction; calculating a first value of the first set of sampling phases; calculating a second value of the second set of sampling phases; and determining the concentration of the antibody based on a function of the first and second values.

Another aspect relates to a fluorescence polarization system for measuring a concentration of an antibody in a sample, the system comprising: a light source; a first polarizing filter causing light emitted from the light source to be polarized in a first polarization direction; a container holding the sample mixed with a fluorescence polarization assay, the container receiving the light polarized in the first polarization direction; movable second and third polarizing filters, the second polarizing filter restricting passage of light fluoresced from within the container to a first polarization component in the first polarization direction, and the third polarizing filter restricting the passage of the light fluoresced from within the container to a second polarization component in a second polarization direction, the second polarization direction being substantially perpendicular to the first polarization direction; a detector for measuring fluorescence polarized in the first polarization direction and fluorescence polarized in the second polarization direction; and a processing circuitry having a memory for storing instructions which, when executed by the processing circuitry, cause the processing circuitry to: perform a measurement cycle measuring a first set of sampling phases of the fluorescence polarized in the first polarization direction and a second set of sampling phases of the fluorescence polarized in the second polarization direction, at least one sampling phase of the fluorescence polarized in the second polarization direction occurring between sampling phases of the fluorescence polarized in the first polarization direction; calculate a first value of the first set of sampling phases; calculate a second value of the second set of sampling phases; and determine the concentration of the antibody based on a function of the first and second values.

Another aspect relates to a method of fluorescence polarization for measuring a concentration of an antibody in a cell sample, the method comprising: performing a measurement cycle of light fluoresced from within the cell sample mixed with a fluorescence polarization assay, the measurement cycle including the following order of measurements: (1) measuring a first sampling phase of fluorescence polarized in a first polarization direction before a midpoint of the measurement cycle; (2) measuring a second sampling phase of fluorescence polarized in a second polarization direction before the midpoint of the measurement cycle, the second polarization direction being perpendicular to the first polarization direction; (3) measuring a third sampling phase of fluorescence polarized in the second polarization direction after the midpoint of the measurement cycle; and (4) measuring a fourth sampling phase of fluorescence polarized in the first polarization direction after the midpoint of the measurement cycle; calculating a first average of the first and fourth sampling phases; calculating a second average of the second and third sampling phases; and determining the concentration of the antibody based on a function of the first and second averages.

Another aspect relates to fluorescence polarization system for measuring a concentration of antibody protein in a cell sample, the system comprising: a processing circuitry having a memory for storing instructions which, when executed by the processing circuitry, cause the processing circuitry to: perform a measurement cycle of light fluoresced from the cell sample, the measurement cycle including the following order of measurements: (1) measuring a first sampling phase of fluorescence polarized in a first polarization direction before a midpoint of the measurement cycle; (2) measuring a second sampling phase of fluorescence polarized in a second polarization direction before the midpoint of the measurement cycle, the second polarization direction being perpendicular to the first polarization direction; (3) measuring a third sampling phase of fluorescence polarized in the second polarization direction after the midpoint of the measurement cycle; and (4) measuring a fourth sampling phase of fluorescence polarized in the first polarization direction after the midpoint of the measurement cycle; calculate a first average of the first and fourth sampling phases; calculate a second average of the second and third sampling phases; and determine the concentration of the antibody based on a function of the first and second averages.

Another aspect relates to a fluorescence polarization system for measuring a concentration of Immunoglobulin G (IgG) in a sample, the system comprising: a processing circuitry having a memory for storing instructions which, when executed by the processing circuitry, cause the processing circuitry to: perform a measurement cycle measuring a first set of sampling phases of fluorescence polarized in a first polarization direction and a second set of sampling phases of the fluorescence polarized in a second polarization direction, the second polarization direction being substantially perpendicular to the first polarization direction, at least one sampling phase of the fluorescence polarized in the second polarization direction occurring between sampling phases of the fluorescence polarized in the first polarization direction, each sampling phase in the first and second sets of sampling phases including voltage measurements of the light from a plurality of light pulses emitted by a light source; obtain a plurality of voltage measurements from each light pulse, wherein the plurality of voltage measurements from each light pulse includes a first set of voltage measurements when the light source is turned on, and a second set of voltage measurements when the light source is turned off; calculate an average of the first set of voltage measurements; calculate an average of the second set of voltage measurements; determine a light pulse differential value for each light pulse by subtracting the average of the second set of voltage measurements from the average of the first set of voltage measurements; determine a voltage value for each sampling phase by calculating an average of the light pulse differential values of the plurality of light pulses in each sampling phase; determine a first voltage value for the first polarization direction by calculating an average of the voltage values in the first set of sampling phases; determine a second voltage value for the second polarization direction by calculating an average of the voltage values in the second set of sampling phases; determine a fluorescence polarization value by subtracting the second voltage value from the first voltage value and dividing by a sum of the first and second voltage values; and determine a concentration value of the IgG by comparing the fluorescence polarization value to fluorescence polarization values obtained from samples of known concentration.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 is an isometric view of an example of a cell analysis system that analyzes cell health of a plurality of cell samples.

FIG. 2 is another isometric view of the cell analysis system of FIG. 1 having a top cover removed from a housing of the cell analysis system.

FIG. 3 is a top view of a work platform supported inside the housing of the cell analysis system of FIG. 1.

FIG. 4 schematically illustrates an example of a titer module supported on the work platform of FIG. 3.

FIG. 5 graphically illustrates an example of a plot showing detected light intensity drift in the titer module of FIG. 4 causing an error in calculating a concentration of antibody.

FIG. 6 graphically illustrates a plot showing simulated fluorescent polarization measurement errors that result from a measurement sequence shown in the plot of FIG. 5.

FIG. 7 schematically illustrates an example of a method of measuring a concentration of antibody in a cell sample that can be performed by the titer module of FIG. 4.

FIG. 8 schematically illustrates an example of a method of performing a measurement cycle in an operation of the method of FIG. 7.

FIG. 9 schematically illustrates an example of a measurement cycle performed in accordance with the method of FIG. 8.

FIG. 10 graphically illustrates an example of a plot showing how the method of FIG. 7 reduces errors in calculating a concentration of antibody in a sample of cells.

FIG. 11 graphically illustrates a plot showing reduced fluorescent polarization measurement errors that result from the measurement cycle of FIGS. 8-10.

FIG. 12 schematically illustrates a method of measuring a sampling phase performed in the measurement cycle of FIGS. 8-10.

FIG. 13 schematically illustrates an example of a controller of the cell analysis system of FIG. 1 that can be used to implement aspects of the titer module of FIG. 4.

### DETAILED DESCRIPTION

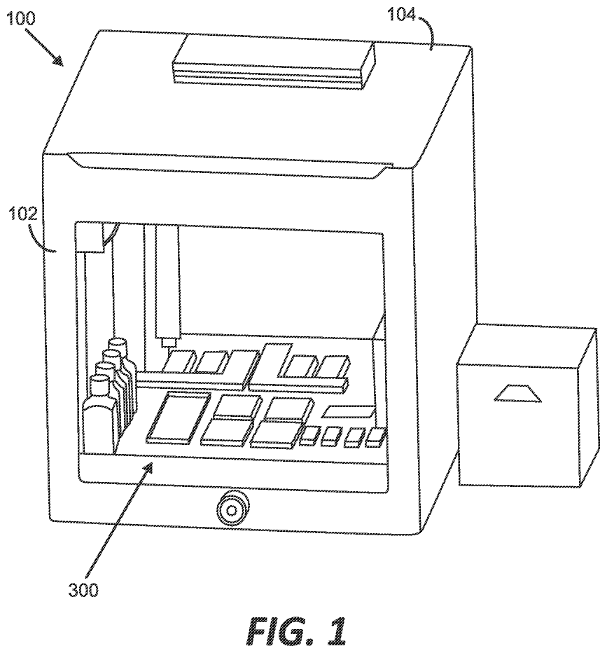

FIG. 1 is an isometric view of an example of a cell analysis system 100 that analyzes cell health of a plurality of cell samples. For a single cell sample, the cell analysis system 100 can measure cell count, cell viability, antibody concentration (e.g., protein titer), and other cell characteristics with minimal interaction from a user of the system.

The cell analysis system 100 includes connectivity to automated bioreactors and other systems and devices. The cell analysis system 100 automates sample preparation, and minimizes sample volume requirements for measuring cell characteristics. The cell analysis system 100 provides remote access to data including the measured cell characteristics, supports multiple users at once, and is compatible with various information technology (IT) structures.

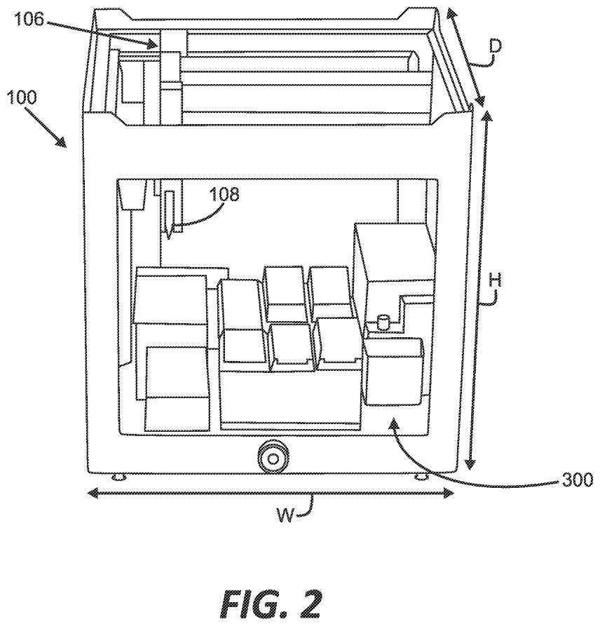

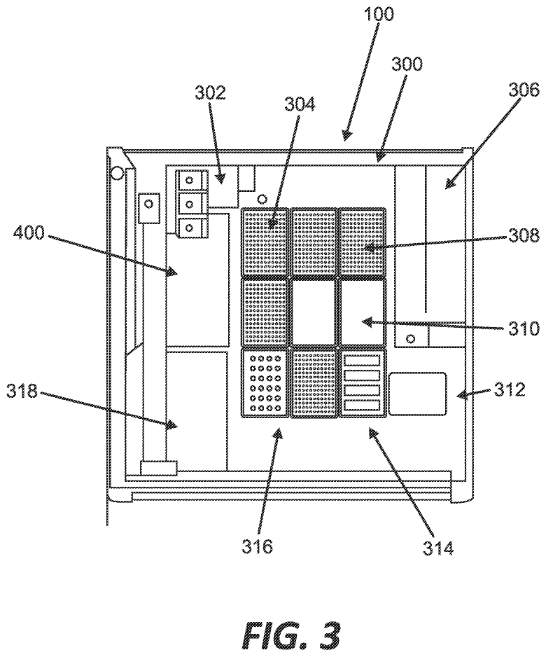

As shown in FIG. 1, the cell analysis system 100 includes a housing 102 that supports a work platform 300. The housing 102 includes a top cover 104 that can support a dispensing system 106 that will be described in more detail with reference to FIGS. 2 and 3.

FIG. 2 is another isometric view of the cell analysis system 100 having the top cover 104 removed from the housing 102, and thereby exposing the dispensing system 106. FIG. 3 is a top view of the work platform 300 supported inside the housing 102. Referring now to FIGS. 2 and 3, the work platform 300 supports one or more tube racks that hold a plurality of containers containing cell samples and various types of reagents. In some instances, at least some of the containers are empty. The dispensing system 106 includes a probe 108 that is movably mounted within the housing 102 in the space above the work platform 300.

The probe 108 is mounted for movement along three mutually perpendicular axes (e.g., X-axis, Y-axis, and Z-axis of a three-dimensional cartesian coordinate system). The three-dimensional movement allows a distal end of the probe 108, through which liquid aspiration and dispensing occurs, to access any container held on the work platform 300 inside the housing 102.

A proximal end of the probe 108 is fluidly connected to a bi-directional pump having a movable actuator that controls the mode of the pump. For example, a first mode can include aspirating and a second mode can include dispensing, and the movable actuator controls switching between the first and second modes, as well as a rate of liquid that is aspirated and dispensed under the first and second modes. As an example, the bi-directional pump can include a syringe pump. Movement of the probe 108 and the movable actuator is controlled by one or more step motors that operate under the control of a controller 1300 that is programmable.

As shown in FIG. 2, the housing 102 is dimensioned to have a width W, a depth D, and a height H. As an illustrative example, the width W is about 24 inches to about 36 inches, the depth D is about 24 inches to about 36 inches, and the height H is about 24 inches to about 36 inches. In some examples, the housing 102 is dimensioned to have a cube shape.

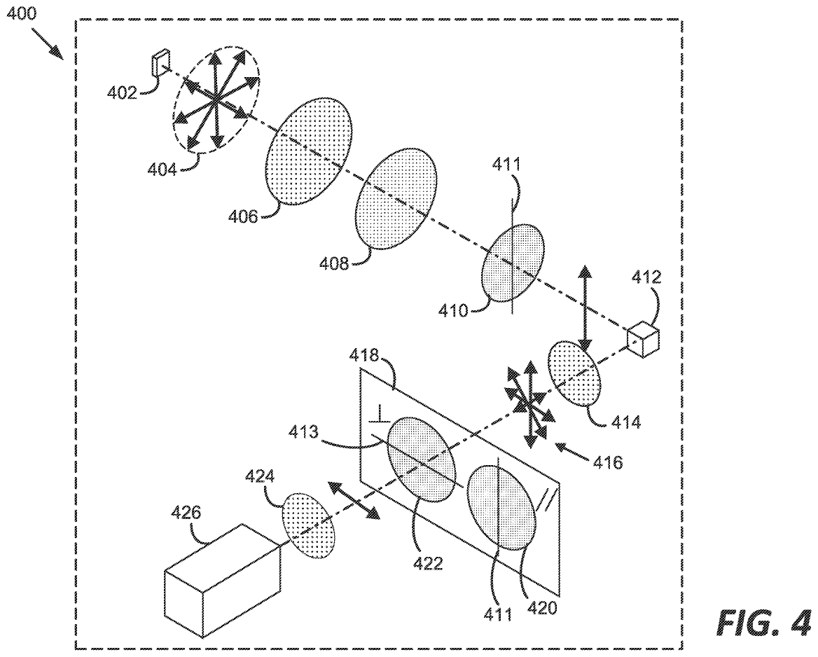

As shown in FIG. 3, the work platform 300 supports a sample transfer module 302, one or more mixing plates 304, a cell health module 306, one or more tip racks 308, one or more titer plates 310, a tip waste bin 312, a diluent reservoir 314, sample inputs 316 such as tube trays and well plates, a metabolite module 318, and a titer module 400. The cell health module 306 measures cell health, including cell count and cell viability. The titer module 400 measures protein titer, and will now be described in more detail with reference to FIG. 4.

FIG. 4 schematically illustrates an example of the titer module 400 supported on the work platform 300. The titer module 400 includes optical components that are used to measure a concentration of an antibody present in a cell sample. For example, the titer module 400 measures a concentration of Immunoglobulin G (IgG) present in a sample of cells.

More specifically, the titer module 400 calculates a fluorescence polarization measurement for measuring the concentration of IgG protein in the sample of cells. Fluorescence polarization includes mixing the sample of cells with a fluorescence polarization assay, and subsequently measuring fluorescence polarization for determining the concentration of the IgG protein in the sample of cells. While the titer module 400 is described herein with reference to measuring IgG concentration, the titer module 400 and the measurement techniques described herein can be used to measure the concentrations of additional types of proteins and antibodies.

As shown in FIG. 4, the titer module 400 includes a light source 402 that emits light 404 toward a container 412 holding a solution of cells mixed with the fluorescence polarization assay. The fluorescence polarization assay binds with antibodies of interest (e.g., IgG) produced by the cells. As discussed above, the dispensing system 106 of the cell analysis system 100 is automated to mix the fluorescence polarization assay together with the sample of cells in the container 412, such that the user of the system does not need to manually mix the solution in the container 412. In some examples, the container 412 is a cuvette, tube, and the like.

The light source 402 emits the light 404 without polarization such that the light 404 is unpolarized light. In some examples, the light source 402 is a light-emitting diode (LED). In some examples, the titer module 400 includes a focusing lens 406 and a spectral filter 408 that respectively focus and filter the light 404 emitted from the light source 402.

The light 404 passes through a first polarizing filter 410 that polarizes the light 404 in a first polarization direction 411. In some examples, the first polarization direction 411 is in a linear direction. In the example shown in FIG. 4, the first polarization direction 411 is vertically linear. In alternative examples, the first polarization direction 411 is horizontally linear. Additional polarization directions are possible.

The light 404, once polarized in the first polarization direction 411, is absorbed by the solution of cells mixed with the fluorescence polarization assay in the container 412. This causes the solution in the container 412 to emit polarized fluorescence light 416.

The polarized fluorescence light 416 can pass through a lens 414 for focusing the polarized fluorescence light 416 after emission from the solution in the container 412. Afterwards, the polarized fluorescence light 416 alternately passes through a second polarizing filter 420 and a third polarizing filter 422. The second and third polarizing filters 420, 422 are mounted to a structure 418 controlled by the controller 1300 to alternate placement of the second and third polarizing filters 420, 422 into the path of the polarized fluorescence light 416.

The second polarizing filter 420 is polarized in a direction parallel to the direction of the first polarizing filter 410 (i.e., in the first polarization direction 411). The second polarizing filter 420 restricts passage of the polarized fluorescence light 416 to the first polarization direction 411.

The third polarizing filter 422 is polarized in a second polarization direction 413 perpendicular to the first polarization direction 411 of the first polarizing filter 410. The third polarizing filter 422 restricts passage of the polarized fluorescence light 416 to the second polarization direction 413.

Thereafter, the polarized fluorescence light 416 passes through a spectral filter 424 before detection by a detector 426. In some examples, the detector 426 a photomultiplier tube (PMT). The detector 426 converts the polarized fluorescence light 416 into voltage values for input into Equation 1 to determine a concentration of antibody produced by the same of cells.

$$FP ( mP ) = \frac{Ipar - Iper}{Ipar + Iper} * 1000 = \frac{1 - Iper / Ipar}{1 + Iper / Ipar} * 1 \; 000 , \qquad ( 1 )$$

where I_{PAR} is a voltage of parallel fluorescence detected from the solution in the container 412, I_{PER} is a voltage of perpendicular fluorescence detected from the solution in the container 412, and FP is fluorescence polarization in milli-polarization units (mP). As an example, the voltages of I_{PAR} and I_{PER} detected by the detector 426 can range from about 0.02 volts to about 5.0 volts.

The measured fluorescence polarization (FP) is correlated with the concentration of antibody in the sample of cells. For example, a higher detected FP correlates to a higher concentration of antibody, and a lower detected FP correlates to a lower concentration of antibody. This is because a higher concentration of antibody will have increased binding with the fluorescence polarization assay such that the voltage of parallel fluorescence is larger, and the voltage of perpendicular fluorescence is smaller, which results in a larger detected FP.

A technical challenge with Equation 1 is that any error in measuring I_{PAR} that is not present in I_{PER} can cause an error in calculation of the fluorescence polarization (FP). For example, the FP calculation depends on the measured ratio I_{PER}/I_{PAR}. Any variation that affects I_{PAR} and I_{PER} proportionally is canceled out. However, errors such as signal intensity variation over the course of measuring the parallel fluorescence (I_{PAR}) and the perpendicular fluorescence (I_{PER}), as well as offset errors affecting both the perpendicular fluorescence (I_{PER}) and the parallel fluorescence (I_{PAR}), do not get canceled out and can cause significant errors in the FP calculation, and thereby causing errors when calculating a concentration of antibody in a sample of cells.

Sources of signal intensity variation can include, without limitation, intensity fluctuation and drift of the light source 402, electrical response, dye bleaching, and non-uniform fluid and diffusion in the container 412. Additional sources of errors are possible. Sources of offset noise can include, without limitation, electrical circuit noise and ambient light.

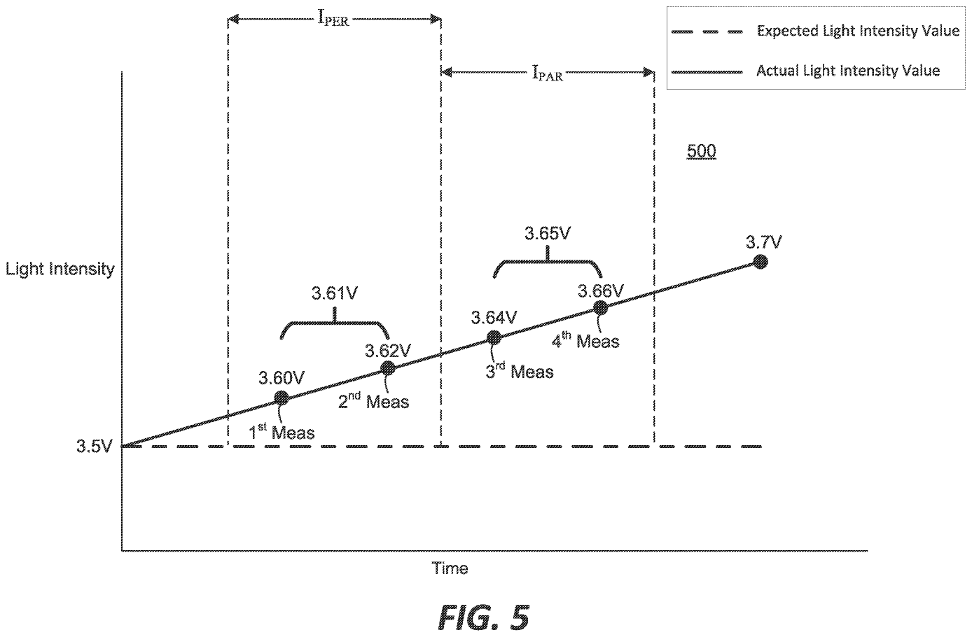

FIG. 5 graphically illustrates an example of a plot 500 showing detected light intensity drift in the titer module 400 causing an error in the FP calculation, and miscalculation of the concentration of antibody in the sample of cells. The plot 500 includes a light intensity (Y-axis) measured by the detector 426 over time (X-axis). In this example, the light intensity is measured without any solution of cells and fluorescence polarization assay in the container 412, such that the drift of the light intensity is not caused by the solution.

In this example, the light intensity of the light source 402 is expected to have a constant value of 3.5V. Thus, the ratio between the I_{PER} and I_{PAR} fluorescence should be 1:1 (e.g., 3.5V: 3.5V) because there is no sample of cells containing antibodies in the container 412.

However, the actual light intensity shown in the plot 500 exhibits a voltage drift that causes an error in the ratio between I_{PER} and I_{PAR} fluorescence. In this example, a measurement cycle is performed where the I_{PER} fluorescence is measured in a first phase (i.e., using the third polarizing filter 422), and thereafter the I_{PAR} fluorescence is measured in a second phase (i.e., using the second polarizing filter 420). Each of the I_{PER} and I_{PAR} fluorescence phases include two measurements. For example, the I_{PER} fluorescence includes a first measurement of 3.60V and a second measurement of 3.62V, and the I_{PAR} fluorescence includes a third measurements of 3.64V and a fourth measurement of 3.66V. The I_{PER} fluorescence is calculated as an average of the first and second measurements (i.e., 3.61V), and the I_{PAR} fluorescence is calculated as an average of the third and fourth measurements (i.e., 3.65V). This results in a ratio of 3.61:3.65 between the I_{PER} and I_{PAR} fluorescence, which is about a 1% error caused by the drift of the light source 402.

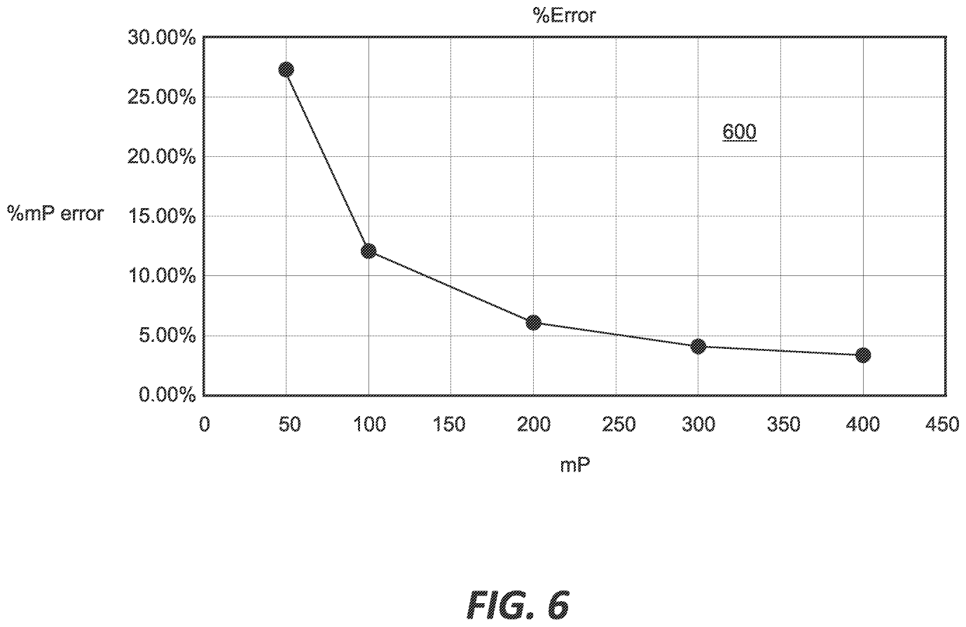

FIG. 6 graphically illustrates a plot 600 showing simulated fluorescent polarization measurement errors that result from the measurement sequence of FIG. 5. As shown in FIG. 6, the measurement errors are significantly greater for lower mP values such as mP values less than 200 units. Thus, lower concentrations of antibodies in the samples held by the container are especially sensitive to signal intensity variation and noise in the titer module 400.

In some instances, measurement errors in the ratio between the I_{PER} and I_{PAR} fluorescence from signal intensity variation and noise are reduced by implementing complicated electrical circuit designs and temperature controlled light sources and detectors in the titer module. However, such solutions are expensive, have low reliability, can increase the size of the titer module, and slow down the warmup time for using the device.

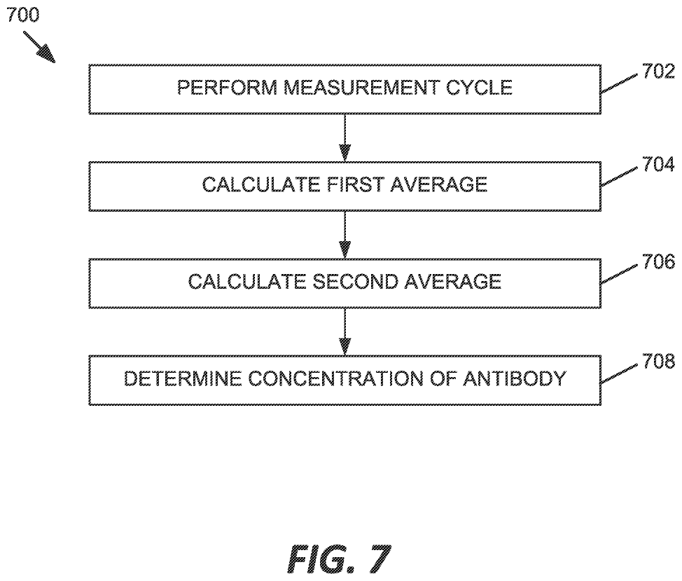

FIG. 7 schematically illustrates an example of a method 700 of measuring a concentration of an antibody in a cell sample. The method 700 uses the optical components of the titer module 400 (shown in FIG. 4) to calculate the concentration of the antibody in the cell sample. In some examples, the method 700 is performed to measure a concentration of Immunoglobulin G (IgG) in a solution containing a fluorescence polarization assay is mixed in the container 412 using the dispensing system 106 of the cell analysis system 100.

The method 700 reduces errors from signal intensity variation and noise without requiring changes to the optical components and hardware of the titer module 400. Instead, the method 700 is a software solution that improves the functioning of the titer module 400 by reducing these types errors to improve the accuracy of the antibody concentration calculation.

The method 700 includes an operation 702 of performing a measurement cycle. Operation 702 includes using the optical components of the titer module 400 to take a first set of measurements of light fluoresced from the solution in the first polarization direction 411, and to take a second set of measurements of light fluoresced from the solution in the second polarization direction 413. The first and second sets of measurements alternate with one another in the measurement cycle.

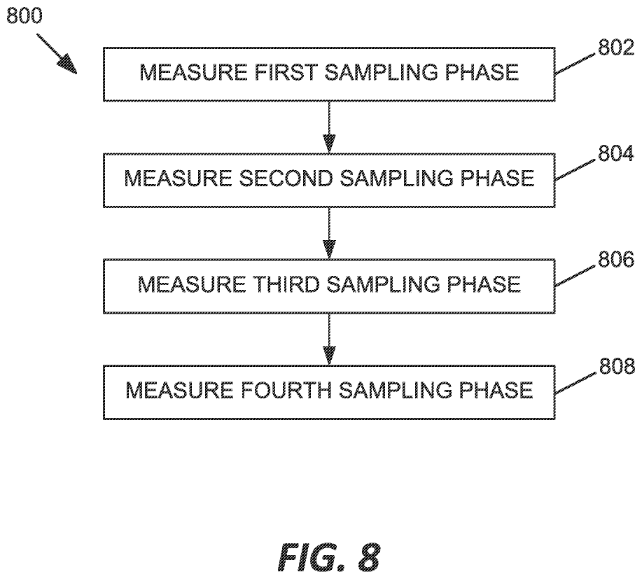

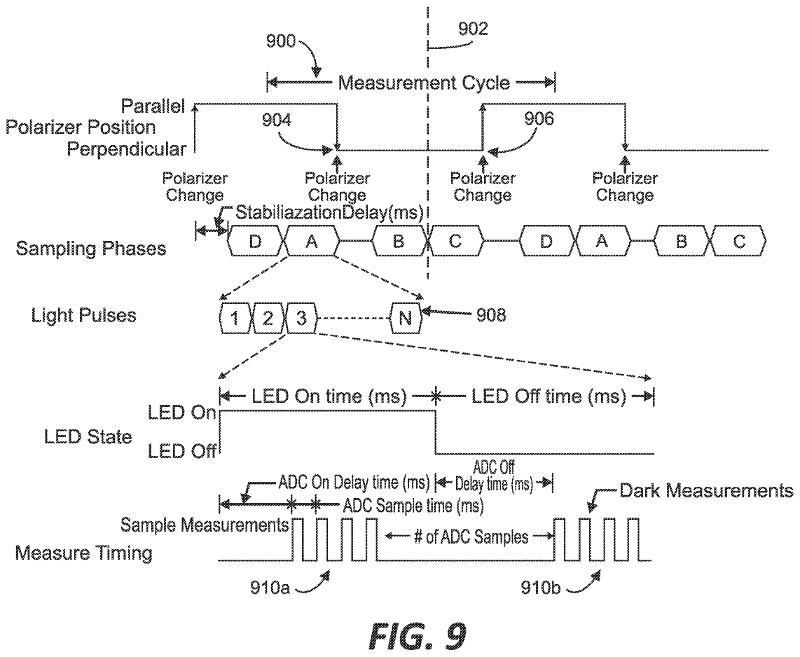

FIG. 8 schematically illustrates an example of a method 800 of performing the measurement cycle in operation 702 of the method 700. FIG. 9 schematically illustrates an example of a measurement cycle 900 performed in accordance with the method 800.

Referring now to FIGS. 8 and 9, the method 800 includes a step 802 of measuring a first sampling phase A of the light fluoresced from the container 412 in the first polarization direction 411 before a midpoint 902 of the measurement cycle 900. Next, the method 800 includes a step 804 of measuring a second sampling phase B of the light fluoresced from the container 412 in the second polarization direction 413 before the midpoint 902 of the measurement cycle 900.

As further shown in FIGS. 8 and 9, the method 800 includes a step 806 of measuring a third sampling phase C of the light fluoresced from the container 412 in the second polarization direction 413 after the midpoint 902 of the measurement cycle 900. Next, the method 800 includes a step 808 of measuring a fourth sampling phase D of the light fluoresced from the container 412 in the first polarization direction 411 after the midpoint 902 of the measurement cycle 900.

The measurement cycle 900 includes at least one sampling phase in the first polarization direction 411 and at least one sampling phase in the second polarization direction 413 before the midpoint, and the measurement cycle includes at least one sampling phase in the first polarization direction 411 and at least one sampling phase in the second polarization direction 413 after the midpoint of the measurement cycle.

During the measurement cycle 900, the structure 418 is controlled by the controller 1300 to switch at a transition point 904 from the second polarizing filter 420 polarized in a direction parallel to the direction of the first polarizing filter 410 to the third polarizing filter 422 that is polarized in a direction perpendicular to the direction of the first polarizing filter 410. The transition point 904 occurs between the first and second sampling phases A, B. Also, during the measurement cycle 900, the structure 418 is controlled by the controller 1300 to switch at a transition point 906 from the third polarizing filter 422 (which is polarized in a direction perpendicular to the direction of the first polarizing filter 410) to the second polarizing filter 420 (which is polarized in a direction parallel to the direction of the first polarizing filter 410). The transition point 906 occurs between the third and fourth sampling phases C, D.

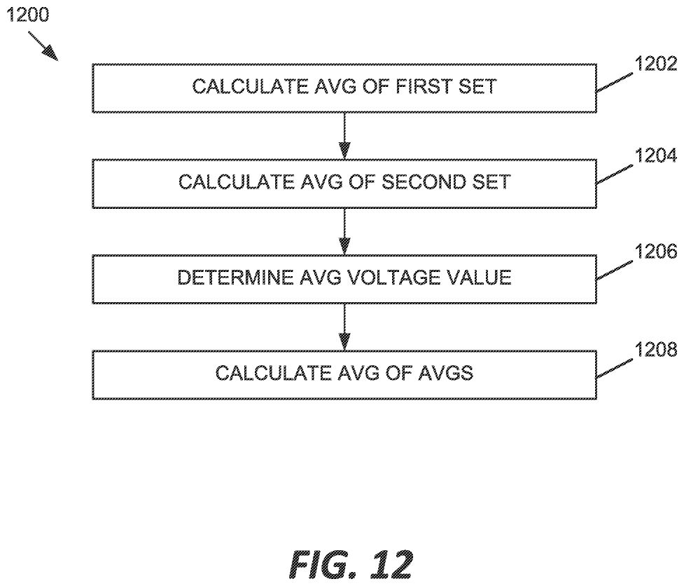

As further shown in FIG. 9, the sampling phases A-D each include voltage measurements of fluoresced light from a plurality of light pulses 908 emitted by the light source 402. For each of the light pulses 908, the detector 426 obtains a plurality of voltage measurements 910. For example, the plurality of voltage measurements 910 from each of the light pulses 908 includes a first set of voltage measurements 910a when the light source 402 is turned on, and a second set of voltage measurements 910b when the light source 402 is turned off. Steps 802-808 for measuring the first, second, third, and fourth sampling phases A, B C, D, respectively, can each include performing additional operations shown in FIG. 12.

FIG. 12. schematically illustrates a method 1200 of measuring a sampling phase performed in the measurement cycle of FIGS. 8-10. Referring now to FIGS. 9 and 12, the method 1200 includes an operation 1202 of calculating an average of the first set of voltage measurements 910a when the light source 402 is turned on. Next, the method 1200 includes an operation 1204 of calculating an average of the second set of voltage measurements 910b when the light source 402 is turned off. Next, the method 1200 includes an operation 1206 of determining a voltage value for each of the light pulses 908 in the sampling phase by subtracting the average of the second set of voltage measurements 910b from the average of the first set of voltage measurements 910a. Next, the method 1200 can include an operation 1208 of determining a voltage value for the sampling phase by calculating an average of the voltage values of the plurality of light pulses 908 in the sampling phase.

Referring back to FIG. 7, the method 700 includes an operation 704 of calculating a first average of the first and fourth sampling phases A, D. As shown in FIG. 9, the first sampling phase A includes light fluoresced in the first polarization direction 411 before the midpoint 902 of the measurement cycle 900. The fourth sampling phase D includes light fluoresced in the first polarization direction 411 after the midpoint 902 of the measurement cycle 900.

Next, the method 700 includes an operation 706 of calculating a second average of the second and third sampling phases B, C. As described above, the second sampling phase B includes light fluoresced from the container in the second polarization direction 413 before the midpoint 902 of the measurement cycle 900. The third sampling phase C includes light fluoresced from the container in the second polarization direction 413 after the midpoint 902 of the measurement cycle 900.

The method 700 includes an operation 708 of determining a concentration of the antibody based on a ratio of the first and second averages. For example, the first average is the voltage of parallel fluorescence (I_{PAR}), the second average is the voltage of perpendicular fluorescence (I_{PER}), and the fluorescence polarization is calculated using Equation 1. The concentration of the antibody is determined by comparing the calculated fluorescence polarization with fluorescence polarization measurements for samples of known antibody concentration.

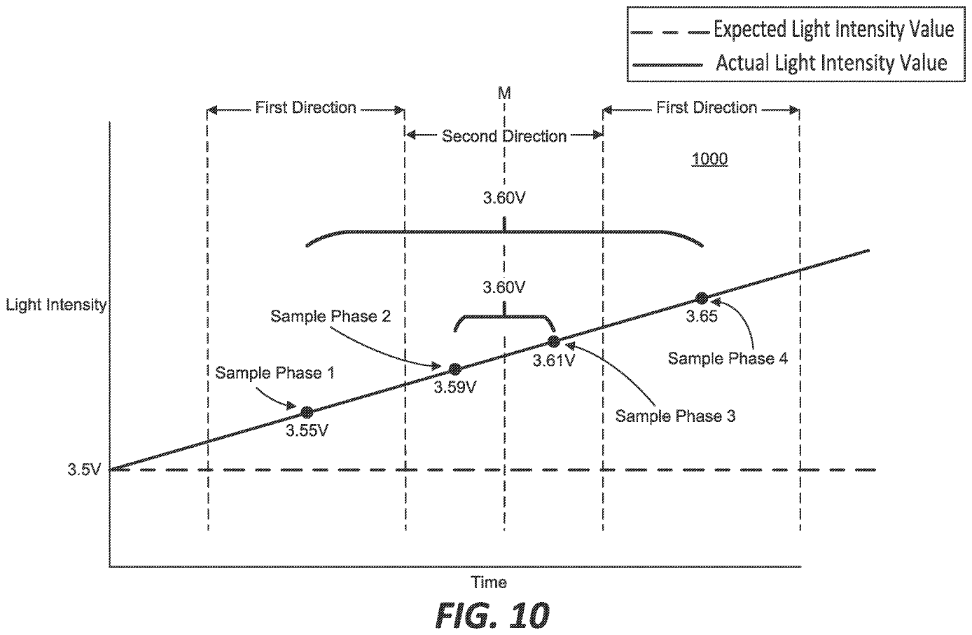

FIG. 10 graphically illustrates an example of a plot 1000 showing how the method 700 eliminates errors in calculating the unit of polarization measurement (mP) used for determining a concentration of antibody in a sample of cells. The plot 1000 includes a light intensity (Y-axis) measured by the detector 426 over time (X-axis). Like in the example plot in FIG. 5, the plot 1000 shows the light source 402 exhibits drift from an expected value of 3.5V. The light intensity is measured without any solution of cells and fluorescence polarization assay in the container 412, such that the drift of the light intensity is not caused by the solution.

As shown in FIG. 10, a measurement cycle is performed where the first sampling phase A is measured for the light fluoresced in the first polarization direction (3.55V) before the midpoint M, the second sampling phase B is measured for the light fluoresced in the second polarization direction (3.59V) before the midpoint M, the third sampling phase C is measured for the light fluoresced in the second polarization direction (3.61V) after the midpoint M, and the fourth sampling phase D is measured for the light fluoresced in the first polarization direction (3.65V) after the midpoint M. The first average of the first and fourth sampling phases A, D is calculated as 3.6V. The second average of the second and third sampling phases B, C is calculated as 3.6V. This results in a ratio of 3.6:3.6 between the I_{PER} and I_{PAR} fluorescence, which eliminates the error caused by the drift of the light source 402 (see, for comparison, the plot 500 shown in FIG. 5). Thus, the measurement cycle performed in the method 800 reduces errors, and even in some instances eliminates the errors, when measuring a concentration of antibody in a cell sample using fluorescence polarization.

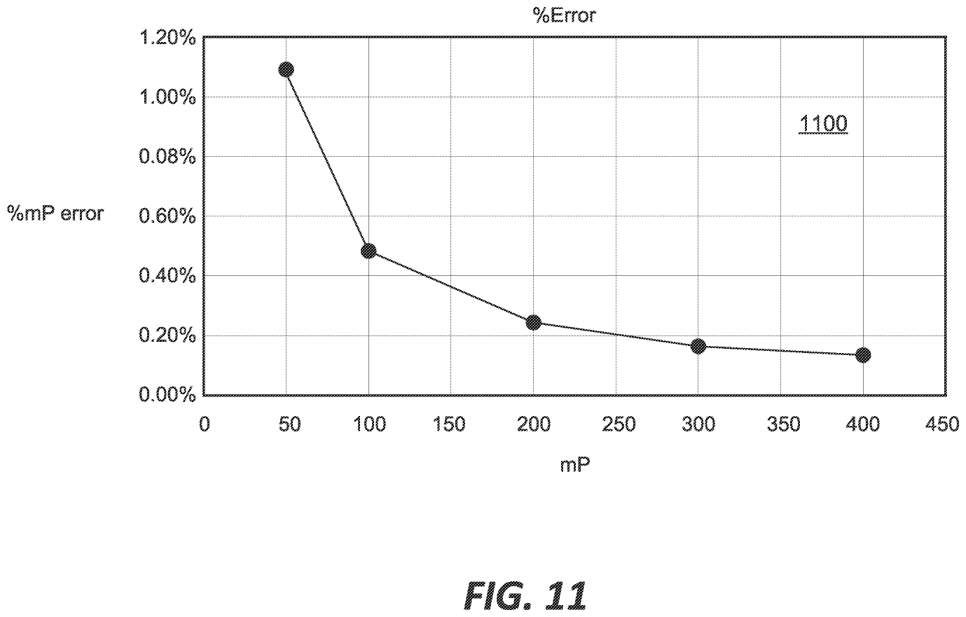

FIG. 11 graphically illustrates a plot 1100 showing a reduction in the simulated fluorescent polarization measurement errors that result from the measurement cycle shown in FIG. 8-10. Referring now to FIGS. 6 and 11, the plot 1100 shows that the measurement errors are significantly less for lower mP values (e.g., mP values less than 200 units) than the measurement errors shown in the plot 600 when a traditional measurement sequence is performed. The plot 1100 shows that sensitivity to signal intensity variation and noise in the titer module 400 is significantly reduced for lower concentrations of antibodies.

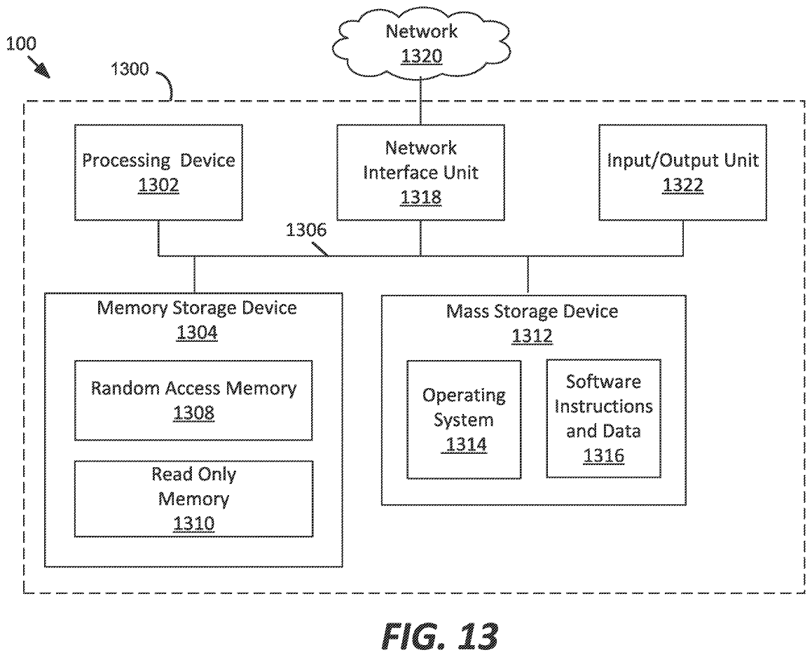

FIG. 13 schematically illustrates an example of the controller 1300 of the cell analysis system 100 that can be used to implement aspects described herein, including the features of the titer module 400. As shown in FIG. 13, the controller 1300 includes one or more processing devices 1302, a memory storage device 1304, and a system bus 1306 that couples the memory storage device 1304 to the one or more processing devices 1302. The one or more processing devices 1302 can include central processing units (CPU). In some instances, the one or more processing devices 1302 are part of a processing circuitry having a memory for storing instructions which, when executed by the processing circuitry, cause the processing circuitry to perform the various aspects, features, and functionalities described herein.

As shown in FIG. 13, the memory storage device 1304 can include a random-access memory (“RAM”) 1308 and a read-only memory (“ROM”) 1310. Basic input and output logic having basic routines that help to transfer information between elements within the controller 1300, such as during startup, can be stored in the ROM 1310.

The controller 1300 can also include a mass storage device 1312 that can include an operating system 1314 and store software instructions and data 1316. The mass storage device 1312 is connected to the processing device 1302 through the system bus 1306. The mass storage device 1312 and associated computer-readable data storage media provide non-volatile, non-transitory storage for the controller 1300.

Although the description of computer-readable data storage media contained herein refers to the mass storage device 1312, it should be appreciated by those skilled in the art that computer-readable data storage media can be any available non-transitory, physical device or article of manufacture from which the controller 1300 can read data and/or instructions. The computer-readable storage media can be comprised of entirely non-transitory media. The mass storage device 1312 is an example of a computer-readable storage device.

Computer-readable data storage media include volatile and non-volatile, removable, and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, or any other medium which can be used to store information, and which can be accessed by the device.

The controller 1300 can operate in a networked environment using logical connections to the other devices through the network 1320. The controller 1300 connects to the network 1320 through a network interface unit 1318 connected to the system bus 1306. The network interface unit 1318 can also connect to additional types of communications networks and devices, including through Bluetooth, Wi-Fi, and cellular telecommunications networks including 4G and 5G networks. The network interface unit 1318 can connect the controller 1300 to additional networks, systems, and devices. The controller 1300 also includes an input/output unit 1322 for receiving and processing inputs and outputs from peripheral devices.

The mass storage device 1312 and the RAM 1308 can store software instructions and data. The software instructions can include an operating system 1314 suitable for controlling the operation of the cell analysis system 100. The mass storage device 1312 and/or the RAM 1308 can also store the software instructions and data 1216, which when executed by the processing device 1302, provide the functionality of the cell analysis system 100 discussed herein.

The various embodiments described above are provided by way of illustration only and should not be construed to be limiting in any way. Various modifications can be made to the embodiments described above without departing from the true spirit and scope of the disclosure.

Embodiments of the disclosure can be described with reference to the following numbered clauses, with preferred features laid out in the dependent clauses:
1. A method of fluorescence polarization for measuring a concentration of an antibody in a sample, the method comprising:
   performing a measurement cycle measuring a first set of sampling phases of fluorescence polarized in a first polarization direction and a second set of sampling phases of the fluorescence polarized in a second polarization direction, at least one sampling phase of the fluorescence polarized in the second polarization direction occurring between sampling phases of the fluorescence polarized in the first polarization direction;
   calculating a first value of the first set of sampling phases;
   calculating a second value of the second set of sampling phases; and
   determining the concentration of the antibody based on a function of the first and second values.
2. The method of clause 1, wherein a first sampling phase of the first set of sampling phases of the fluorescence polarized in the first polarization direction occurs before a midpoint of the measurement cycle and a second sampling phase of the first set of sampling phases of the fluorescence polarized in the first polarization direction occurs after a midpoint of the measurement cycle.
3. The method of clause 1, wherein each sampling phase includes voltage measurements of fluoresced light from a plurality of light pulses emitted by a light source.
4. The method of clause 3, further comprising:
   obtaining a plurality of voltage measurements from each light pulse.
5. The method of clause 4, wherein the plurality of voltage measurements from each light pulse includes a first set of voltage measurements when the light source is turned on, and a second set of voltage measurements when the light source is turned off.
6. The method of clause 5, further comprising:
   calculating a first average of the first set of voltage measurements;
   calculating a second average of the second set of voltage measurements; and
   determining a light pulse differential value for each light pulse by subtracting the second average from the first average.
7. The method of clause 6, further comprising:
   determining an average light pulse differential for a sampling phase by calculating an average of the light pulse differentials from the sampling phase.
8. The method of clause 7, further comprising:
   determining a first voltage value for the first polarization direction by calculating an average of the average light pulse differentials for the first set of sampling phases of the fluorescence polarized in the first polarization direction; and determining a second voltage value for the second polarization direction by calculating an average of the average light pulse differentials for the first set of sampling phases of the fluorescence polarized in the first polarization direction.
9. The method of clause 8, further comprising:
   determining a fluorescence polarization value by subtracting the second voltage value from the first voltage value, and dividing by a sum of the first and second voltage values
10. The method of clause 9, further comprising:
   determining a concentration value by comparing the fluorescence polarization value to fluorescence polarization values obtained from samples of known concentration.
11. The method of clause 1, wherein the antibody is Immunoglobulin G (IgG).
12. A fluorescence polarization system for measuring a concentration of an antibody in a sample, the system comprising:
   a light source;
   a first polarizing filter causing light emitted from the light source to be polarized in a first polarization direction;
   a container holding the sample mixed with a fluorescence polarization assay, the container receiving the light polarized in the first polarization direction;
   movable second and third polarizing filters, the second polarizing filter restricting passage of light fluoresced from within the container to a first polarization component in the first polarization direction, and the third polarizing filter restricting the passage of the light fluoresced from within the container to a second polarization component in a second polarization direction, the second polarization direction being substantially perpendicular to the first polarization direction;
   a detector for measuring fluorescence polarized in the first polarization direction and fluorescence polarized in the second polarization direction; and
   a processing circuitry having a memory for storing instructions which, when executed by the processing circuitry, cause the processing circuitry to:
      perform a measurement cycle measuring a first set of sampling phases of the fluorescence polarized in the first polarization direction and a second set of sampling phases of the fluorescence polarized in the second polarization direction, at least one sampling phase of the fluorescence polarized in the second polarization direction occurring between sampling phases of the fluorescence polarized in the first polarization direction;
      calculate a first value of the first set of sampling phases;
      calculate a second value of the second set of sampling phases; and
      determine the concentration of the antibody based on a function of the first and second values.
13. The system of clause 12, wherein a first sampling phase of the first set of sampling phases of the fluorescence polarized in the first polarization direction occurs before a midpoint of the measurement cycle and a second sampling phase of the first set of sampling phases of the fluorescence polarized in the first polarization direction occurs after a midpoint of the measurement cycle.
14. The system of clause 13, wherein each sampling phase includes voltage measurements of fluoresced light from a plurality of light pulses emitted by the light source.
15. The system of clause 14, wherein the instructions, when executed by the processing circuitry, further cause the processing circuitry to:
   obtain a plurality of voltage measurements from each light pulse.
16. The system of clause 15, wherein the plurality of voltage measurements from each light pulse includes a first set of voltage measurements when the light source is turned on, and a second set of voltage measurements when the light source is turned off.
17. The system of clause 16, wherein the instructions, when executed by the processing circuitry, further cause the processing circuitry to:
   calculate a first average of the first set of voltage measurements;
   calculate a second average of the second set of voltage measurements; and
   determining a light pulse differential value for each light pulse by subtracting the second average from the first average.
18. The system of clause 17, wherein the instructions, when executed by the processing circuitry, further cause the processing circuitry to:
   determine an average light pulse differential for a sampling phase by calculating an average of the light pulse differentials from the sampling phase.
19. The system of clause 18, wherein the instructions, when executed by the processing circuitry, further cause the processing circuitry to:
   determine a first voltage value for the first polarization direction by calculating an average of the average light pulse differentials for the first set of sampling phases of the fluorescence polarized in the first polarization direction; and determine a second voltage value for the second polarization direction by calculating an average of the average light pulse differentials for the second set of sampling phases of the fluorescence polarized in the second polarization direction.
20. The system of clause 19, wherein the instructions, when executed by the processing circuitry, further cause the processing circuitry to:
   determine a fluorescence polarization value by subtracting the second voltage value from the first voltage value, and dividing by a sum of the first and second voltage values
21. The system of clause 20, wherein the instructions, when executed by the processing circuitry, further cause the processing circuitry to:
   determine a concentration value by comparing the fluorescence polarization value to fluorescence polarization values obtained from samples of known concentration.
22. The system of clause 12, wherein the antibody is Immunoglobulin G (IgG).
23. A method of fluorescence polarization for measuring a concentration of an antibody in a cell sample, the method comprising:
   performing a measurement cycle of light fluoresced from within the cell sample mixed with a fluorescence polarization assay, the measurement cycle including the following order of measurements:
      (1) measuring a first sampling phase of fluorescence polarized in a first polarization direction before a midpoint of the measurement cycle;
      (2) measuring a second sampling phase of fluorescence polarized in a second polarization direction before the midpoint of the measurement cycle, the second polarization direction being perpendicular to the first polarization direction;
      (3) measuring a third sampling phase of fluorescence polarized in the second polarization direction after the midpoint of the measurement cycle; and
      (4) measuring a fourth sampling phase of fluorescence polarized in the first polarization direction after the midpoint of the measurement cycle;
   calculating a first average of the first and fourth sampling phases;
   calculating a second average of the second and third sampling phases; and
   determining the concentration of the antibody based on a function of the first and second averages.
24. The method of clause 23, wherein each sampling phase includes voltage measurements of fluoresced light from a plurality of light pulses emitted by a light source.
25. The method of clause 24, further comprising:
   obtaining a plurality of voltage measurements from each light pulse.
26. The method of clause 25, wherein the plurality of voltage measurements from each light pulse includes a first set of voltage measurements when the light source is turned on, and a second set of voltage measurements when the light source is turned off.
27. The method of clause 26, further comprising:
   calculating an average of the first set of voltage measurements;
   calculating an average of the second set of voltage measurements; and
   determining a light pulse differential value for each light pulse by subtracting the average of the second set of voltage measurements from the average of the first set of voltage measurements.
28. The method of clause 27, further comprising:
   determining an average voltage value for each sampling phase by calculating an average of the light pulse differential values of the plurality of light pulses in each sampling phase.
29. The method of clause 28, further comprising:
   determining a first voltage value for the first polarization direction by calculating an average of the average voltage values for the first and fourth sampling phases; and
   determining a second voltage value for the second polarization direction by calculating an average of the average voltage values for the second and third sampling phases.
30. The method of clause 29, further comprising:
   determining a fluorescence polarization value by subtracting the second voltage value from the first voltage value, and dividing by a sum of the first and second voltage values.
31. The method of clause 30, further comprising:
   determining a concentration value by comparing the fluorescence polarization value to fluorescence polarization values obtained from samples of known concentration.
32. The method of clause 23, wherein the antibody is Immunoglobulin G (IgG).
33. A fluorescence polarization system for measuring a concentration of antibody protein in a cell sample, the system comprising:
   a processing circuitry having a memory for storing instructions which, when executed by the processing circuitry, cause the processing circuitry to:
      perform a measurement cycle of light fluoresced from the cell sample, the measurement cycle including the following order of measurements:
      (1) measuring a first sampling phase of fluorescence polarized in a first polarization direction before a midpoint of the measurement cycle;
      (2) measuring a second sampling phase of fluorescence polarized in a second polarization direction before the midpoint of the measurement cycle, the second polarization direction being perpendicular to the first polarization direction;
      (3) measuring a third sampling phase of fluorescence polarized in the second polarization direction after the midpoint of the measurement cycle; and
      (4) measuring a fourth sampling phase of fluorescence polarized in the first polarization direction after the midpoint of the measurement cycle;
   calculate a first average of the first and fourth sampling phases;
   calculate a second average of the second and third sampling phases; and
   determine the concentration of the antibody based on a function of the first and second averages.
34. The system of clause 33, wherein each sampling phase includes voltage measurements of fluoresced light from a plurality of light pulses emitted by a light source.
35. The system of clause 34, wherein the instructions, when executed by the processing circuitry, further cause the processing circuitry to:
   obtain a plurality of voltage measurements from each light pulse.
36. The system of clause 35, wherein the plurality of voltage measurements from each light pulse includes a first set of voltage measurements when the light source is turned on, and a second set of voltage measurements when the light source is turned off.
37. The system of clause 36, wherein the instructions, when executed by the processing circuitry, further cause the processing circuitry to:
   calculate an average of the first set of voltage measurements;
   calculate an average of the second set of voltage measurements; and
   determine a light pulse differential value for each light pulse by subtracting the average of the second set of voltage measurements from the average of the first set of voltage measurements.
38. The system of clause 37, wherein the instructions, when executed by the processing circuitry, further cause the processing circuitry to:
   determine a voltage value for each sampling phase by calculating an average of the light pulse differential values of the plurality of light pulses in each sampling phase.
39. The system of clause 38, wherein the instructions, when executed by the processing circuitry, further cause the processing circuitry to:
   determine a first voltage value for first polarization direction by calculating an average of the voltage values for the first and fourth sampling phases; and
   determine a second voltage value for the second polarization direction by calculating an average of the voltage values for the second and third sampling phases.
40. The system of clause 39, wherein the instructions, when executed by the processing circuitry, further cause the processing circuitry to:
   determine a fluorescence polarization value by subtracting the second voltage value from the first voltage value, and dividing by a sum of the first and second voltage values.
41. The system of clause 40, wherein the instructions, when executed by the processing circuitry, further cause the processing circuitry to:
   determine the concentration of the antibody by comparing the fluorescence polarization value to fluorescence polarization values obtained from samples of known concentration.
42. The system of clause 33, wherein the antibody is Immunoglobulin G (IgG).
43. A fluorescence polarization system for measuring a concentration of Immunoglobulin G (IgG) in a sample, the system comprising:
   a processing circuitry having a memory for storing instructions which, when executed by the processing circuitry, cause the processing circuitry to:
      perform a measurement cycle measuring a first set of sampling phases of fluorescence polarized in a first polarization direction and a second set of sampling phases of the fluorescence polarized in a second polarization direction, the second polarization direction being substantially perpendicular to the first polarization direction, at least one sampling phase of the fluorescence polarized in the second polarization direction occurring between sampling phases of the fluorescence polarized in the first polarization direction, each sampling phase in the first and second sets of sampling phases including voltage measurements of the light from a plurality of light pulses emitted by a light source;
      obtain a plurality of voltage measurements from each light pulse, wherein the plurality of voltage measurements from each light pulse includes a first set of voltage measurements when the light source is turned on, and a second set of voltage measurements when the light source is turned off;
      calculate an average of the first set of voltage measurements;
      calculate an average of the second set of voltage measurements;
      determine a light pulse differential value for each light pulse by subtracting the average of the second set of voltage measurements from the average of the first set of voltage measurements;
      determine a voltage value for each sampling phase by calculating an average of the light pulse differential values of the plurality of light pulses in each sampling phase;
      determine a first voltage value for the first polarization direction by calculating an average of the voltage values in the first set of sampling phases;
      determine a second voltage value for the second polarization direction by calculating an average of the voltage values in the second set of sampling phases;
      determine a fluorescence polarization value by subtracting the second voltage value from the first voltage value and dividing by a sum of the first and second voltage values; and
      determine a concentration value of the IgG by comparing the fluorescence polarization value to fluorescence polarization values obtained from samples of known concentration.

1. A method of fluorescence polarization for measuring a concentration of an antibody in a sample, the method comprising:
performing a measurement cycle measuring a first set of sampling phases of fluorescence polarized in a first polarization direction, with at least two sampling phases of fluorescence polarized in the first polarization direction in the first set occurring about a midpoint of the measurement cycle, and a second set of sampling phases of the fluorescence polarized in a second polarization direction, at least one sampling phase of the fluorescence polarized in the second polarization direction occurring between sampling phases of the fluorescence polarized in the first polarization direction;
calculating a first value of the first set of sampling phases;
calculating a second value of the second set of sampling phases; and
determining the concentration of the antibody based on a function of the first and second values.

2. The method of claim 1, wherein a first sampling phase of the first set of sampling phases of the fluorescence polarized in the first polarization direction occurs before the midpoint of the measurement cycle and a second sampling phase of the first set of sampling phases of the fluorescence polarized in the first polarization direction occurs after the midpoint of the measurement cycle.

3. The method of claim 1, wherein each sampling phase includes voltage measurements of fluoresced light from a plurality of light pulses emitted by a light source, and the method further comprises:
obtaining a plurality of voltage measurements from each light pulse, wherein the plurality of voltage measurements from each light pulse includes a first set of voltage measurements when the light source is turned on, and a second set of voltage measurements when the light source is turned off.

4. The method of claim 3, further comprising:
calculating a first average of the first set of voltage measurements;
calculating a second average of the second set of voltage measurements; and
determining a light pulse differential value for each light pulse by subtracting the second average from the first average.

5. The method of claim 4, further comprising:
determining an average light pulse differential for a sampling phase by calculating an average of the light pulse differentials from the sampling phase.

6. The method of claim 5, further comprising:
determining a first voltage value for the first polarization direction by calculating an average of the average light pulse differentials for the first set of sampling phases of the fluorescence polarized in the first polarization direction; and
determining a second voltage value for the second polarization direction by calculating an average of the average light pulse differentials for the first set of sampling phases of the fluorescence polarized in the first polarization direction.

7. The method of claim 6, further comprising:
determining a fluorescence polarization value by subtracting the second voltage value from the first voltage value, and dividing by a sum of the first and second voltage values.

8. The method of claim 7, further comprising:
determining a concentration value by comparing the fluorescence polarization value to fluorescence polarization values obtained from samples of known concentration.

9. A fluorescence polarization system for measuring a concentration of an antibody in a sample, the system comprising:
a light source;
a first polarizing filter causing light emitted from the light source to be polarized in a first polarization direction;
a container holding the sample mixed with a fluorescence polarization assay, the container receiving the light polarized in the first polarization direction;
movable second and third polarizing filters, the second polarizing filter restricting passage of light fluoresced from within the container to a first polarization component in the first polarization direction, and the third polarizing filter restricting the passage of the light fluoresced from within the container to a second polarization component in a second polarization direction, the second polarization direction being substantially perpendicular to the first polarization direction;
a detector for measuring fluorescence polarized in the first polarization direction and fluorescence polarized in the second polarization direction; and
a processing circuitry having a memory for storing instructions which, when executed by the processing circuitry, cause the processing circuitry to:
perform a measurement cycle measuring a first set of sampling phases of the fluorescence polarized in the first polarization direction, with at least two sampling phases of fluorescence polarized in the first polarization direction in the first set occurring about a midpoint of the measurement cycle, and a second set of sampling phases of the fluorescence polarized in the second polarization direction, at least one sampling phase of the fluorescence polarized in the second polarization direction occurring between sampling phases of the fluorescence polarized in the first polarization direction;
calculate a first value of the first set of sampling phases;
calculate a second value of the second set of sampling phases; and
determine the concentration of the antibody based on a function of the first and second values.

10. The system of claim 9, wherein a first sampling phase of the first set of sampling phases of the fluorescence polarized in the first polarization direction occurs before the midpoint of the measurement cycle and a second sampling phase of the first set of sampling phases of the fluorescence polarized in the first polarization direction occurs after the midpoint of the measurement cycle.

11. The system of claim 10, wherein each sampling phase includes voltage measurements of fluoresced light from a plurality of light pulses emitted by the light source; and
wherein the instructions, when executed by the processing circuitry, further cause the processing circuitry to:
obtain a plurality of voltage measurements from each light pulse, wherein the plurality of voltage measurements from each light pulse includes a first set of voltage measurements when the light source is turned on, and a second set of voltage measurements when the light source is turned off.

12. The system of claim 11, wherein the instructions, when executed by the processing circuitry, further cause the processing circuitry to:
calculate a first average of the first set of voltage measurements;
calculate a second average of the second set of voltage measurements; and
determining a light pulse differential value for each light pulse by subtracting the second average from the first average.

13. The system of claim 12, wherein the instructions, when executed by the processing circuitry, further cause the processing circuitry to:
determine an average light pulse differential for a sampling phase by calculating an average of the light pulse differentials from the sampling phase.

14. The system of claim 13, wherein the instructions, when executed by the processing circuitry, further cause the processing circuitry to:
determine a first voltage value for the first polarization direction by calculating an average of the average light pulse differentials for the first set of sampling phases of the fluorescence polarized in the first polarization direction; and
determine a second voltage value for the second polarization direction by calculating an average of the average light pulse differentials for the second set of sampling phases of the fluorescence polarized in the second polarization direction.

15. The system of claim 14, wherein the instructions, when executed by the processing circuitry, further cause the processing circuitry to:
determine a fluorescence polarization value by subtracting the second voltage value from the first voltage value, and dividing by a sum of the first and second voltage values; and
determine a concentration value by comparing the fluorescence polarization value to fluorescence polarization values obtained from samples of known concentration.
